(19) United States
(12) Patent Application Publication
(10) Pub. No.: US 2025/0246283 A1
(43) Pub. Date: Jul. 31, 2025

(54) METHOD AND APPARATUS FOR PROVIDING SUGGESTIONS FOR MAINTAINING PERSONAL HYGIENE OF A USER

(71) Applicant: Samsung Electronics Co., Ltd., Suwon-si (KR)

(72) Inventors: Dhwanit CHAUHAN, Noida (IN); Aayushi MITTAL, Noida (IN); Vinay Kumar GUPTA, Noida (IN); Jayadhar GANDHAM, Noida (IN); Amit KUMAR, Noida (IN)

(21) Appl. No.: 19/063,970

(22) Filed: Feb. 26, 2025

Related U.S. Application Data
(63) Continuation of application No. PCT/KR2022/017972, filed on Nov. 15, 2022.

(30) Foreign Application Priority Data
Sep. 23, 2022 (IN) 202211054642

(51) Int. Cl. G16H 20/00

(52) U.S. Cl. CPC G16H 20/00

(57) ABSTRACT

A method for providing suggestions for maintaining personal hygiene of a user is provided. The method includes receiving hygiene data associated with the user from at least one source in response to detecting an activity, determining a hygiene category of the activity among a plurality of hygiene categories based on the hygiene data, and preprocessed hygiene data associated with the user, determining a type of the activity among a plurality of types of activities based on the hygiene data and the hygiene category, determining an impact level of the activity on the personal hygiene of the user based on the hygiene category, and the type of the activity, and providing one or more suggestions for maintaining the personal hygiene to the user based on the impact level, the hygiene data, the hygiene category, and the activity.

### CROSS-REFERENCE TO RELATED APPLICATION(S)

This application is a continuation application, claiming priority under § 365(c), of an International application No. PCT/KR2022/017972, filed on Nov. 15, 2022, which is based on and claims the benefit of an Indian Patent Application number 202211054642, filed on Sep. 23, 2022, in the Indian Intellectual Property Office, the disclosure of which is incorporated by reference herein in its entirety.

### BACKGROUND

### 1. Field

The disclosure relates to personal hygiene maintenance. More particularly, the disclosure relates to a method and a system for providing one or more suggestions for maintaining a personal hygiene of a user.

### 2. Description of Related Art

Traditionally, personal hygiene includes behaviors or habits that must be practiced in daily life, starting from morning to night till one goes to sleep to protect both personal health and public image.

The body performs better when it is clean and healthy. Good personal hygiene should include clean skin, oral care, hair care, and body care (sanitization).

It is easy to skip such activities which can lead to health problems or might create problems for a user outside in various public places such as an educational institute, a workplace or the like.

A survey was conducted on school children and retrospective cross-sectional study brings together data on 2,035 schoolchildren recruited from thirty schools. Analysis of the personal hygiene concluded that the hygiene components showcased were poor in 91, 72 and 67% of students, respectively.

To assess the personal hygiene and sanitary condition of an area, a cross sectional study was done in the area. A total of 80 households were randomly selected from two wards of the area. 64% had good personal hygiene practice and 36% did not have good personal hygiene.

A solution according to the related art includes a method for use while assisting user in focusing on performing actions related to personal hygiene care. The method includes collecting data from one or more sensors associated with the user during personal hygiene activities. Further, the method includes processing the collected data to detect at least one feature indicative of a distracting event for the user when performing personal hygiene activities. For example, the collected data is monitored and can be compared against features indicative of distraction events stored in memory to detect when at least one characteristic indicative of a distracting event for the user while performing personal hygiene activities. Further, one or more of a plurality of actions associated with at least one detected characteristic may be selected.

The solution according to the related art identifies personal hygiene activities and detects any distraction being caused only when the personal hygiene activities are being performed. Sub activities like brushing, flossing etc. which are supposed to increase personal hygiene of a user are identified in the related art and the related art focuses on any distractions like calls, messages etc. which may prevent the user from performing these activities appropriately. The solution according to the related art includes distractions hindering the performance of personal hygiene activities. The main intent of the related art is to avoid user losing focus at the time of performing any physical hygiene activity.

Another solution according to the related art includes a method for enhanced personal care with linear data combining attributes. The method includes maintaining an interactive diary for a user configured to collect information regarding personal care assessed for the user over a period of time using at least one personal care module.

In general, information about an individual's personal care may be obtained at a personal care device.

The device may collect information and may provide the information to a remote device arrangement, including but not limited to the internet “cloud” and may process the information received. An interactive diary may be provided that tracks attributes of each user and may process data. Furthermore, the personal care device may be enabled to make recommendations related to and based upon the information collected regarding personal care, such as determining the user's body type and recommending clothing for the user, determining skin tone and making a recommendation for particular skin care and/or cosmetic purchases, and so forth. Prior preferences may be recognized using the interactive diary and certain options may be offered to the user based on past history or indicated learned preferences. The other solution according to the related art may utilize user data, but an impact is not calculated as to how a suggestion may improve the user's personal hygiene. The other solution according to the related art further fails to disclose a feedback system to confirm whether user performed the action suggested.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide a method and system for providing suggestions for maintaining personal hygiene of a user.

Additional aspects will be set forth in part in the description which follows and, in part, will be apparent from the description, or may be learned by practice of the presented embodiments.

In accordance with an aspect of the disclosure, a method for providing, by an electronic device, one or more suggestions for maintaining personal hygiene of a user is provided. The method includes receiving hygiene data associated with the user from at least one source in response to detecting an activity, determining a hygiene category of the activity among a plurality of hygiene categories based on the hygiene data, and preprocessed hygiene data associated with the user, determining a type of the activity among a plurality of types of activities based on the hygiene data and the hygiene category, determining an impact level of the activity on the personal hygiene of the user based on the hygiene category, and the type of the activity, and providing one or more suggestions for maintaining the personal hygiene to the user based on the impact level, the hygiene data, the hygiene category, and the activity.

In accordance with another aspect of the disclosure, an electronic device for providing one or more suggestions for maintaining personal hygiene of a user is provided. The electronic device includes memory storing one or more computer programs, and one or more processors communicatively coupled to the memory, wherein the one or more computer programs include computer-executable instructions that, when executed by the one or more processors individually or collectively, cause the electronic device to receive hygiene data associated with the user from at least one source in response to detecting an activity, determine a hygiene category of the activity among a plurality of hygiene categories based on the hygiene data, and preprocessed hygiene data associated with the user, determine a type of the activity among a plurality of types of activities based on the hygiene data and the hygiene category, determine an impact level of the activity on the personal hygiene of the user based on the hygiene category, and the type of the activity, and provide one or more suggestions for maintaining the personal hygiene to the user based on the impact level, the hygiene data, the hygiene category, and the activity.

In accordance with another aspect of the disclosure, one or more non-transitory computer readable storage media storing one or more computer programs including computer-executable instructions that, when executed by one or more processors of an electronic device individually or collectively, cause the electronic device to execute operations is provided. The operations include receiving hygiene data associated with the user from at least one source in response to detecting an activity, determining a hygiene category of the activity among a plurality of hygiene categories based on the hygiene data, and preprocessed hygiene data associated with the user, determining a type of the activity among a plurality of types of activities based on the hygiene data and the hygiene category, determining an impact level of the activity on the personal hygiene of the user based on the hygiene category, and the type of the activity, and providing one or more suggestions for maintaining the personal hygiene to the user based on the impact level, the hygiene data, the hygiene category, and the activity.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 illustrates a block diagram depicting a method for providing one or more suggestions for maintaining personal hygiene of a user, according to an embodiment of the disclosure;

FIG. 2 illustrates a schematic block diagram of a system providing one or more suggestions for maintaining personal hygiene of a user, according to an embodiment of the disclosure;

FIG. 3 illustrates an operational flow diagram depicting a process for providing one or more suggestions for maintaining personal hygiene of a user, according to an embodiment of the disclosure;

FIG. 4 illustrates an architectural diagram depicting a method for providing one or more suggestions for maintaining personal hygiene of a user, according to an embodiment of the disclosure;

FIG. 5 illustrates an operational flow diagram depicting a process to receive hygiene data, according to an embodiment of the disclosure;

FIG. 6 illustrates an operational flow diagram depicting a process for detecting a hygiene category related to an activity performed by a user, according to an embodiment of the disclosure;

FIG. 7A illustrates an operational flow diagram depicting a process for determining a type of hygiene activity performed by a user, according to an embodiment of the disclosure;

FIG. 7B illustrates an operational flow diagram depicting a process for determining a type of activity associated with an oral hygiene category, according to an embodiment of the disclosure;

FIG. 7C illustrates an operational flow diagram depicting a process for determining a type of activity associated with a skin hygiene category, according to an embodiment of the disclosure;

FIG. 8 illustrates an operational flow diagram depicting a process for determining an impact level of an activity on a personal hygiene of the user, according to an embodiment of the disclosure;

FIG. 9 illustrates an operational flow diagram depicting a process for determining one or more suggestions, according to an embodiment of the disclosure;

FIG. 10A illustrates a use case diagram depicting determination of a hygiene category related to an activity performed by a user, according to an embodiment of the disclosure;

FIG. 10B illustrates a use case diagram depicting determination of a type of the activity performed by a user, according to an embodiment of the disclosure;

FIG. 10C illustrates a use case diagram depicting determination of an impact on personal hygiene of a user, according to an embodiment of the disclosure;

FIG. 10D illustrates a use case diagram depicting determination of one or more possible suggestions for maintaining the personal hygiene of the user, according to an embodiment of the disclosure;

FIG. 10E illustrates a use case diagram depicting determination of one or more suggestions from the one or more suggestions for maintaining the personal hygiene of the user, according to an embodiment of the disclosure;

FIG. 11 illustrates a use case scenario for suggesting one or more suggestions to maintain oral hygiene to a user, according to an embodiment of the disclosure;

FIG. 12 illustrates a use case scenario for suggesting one or more suggestions to maintain skin hygiene to a user, according to an embodiment of the disclosure; and

FIG. 13 illustrates a use case scenario for suggesting one or more suggestions to maintain skin hygiene, according to an embodiment of the disclosure.

The same reference numerals are used to represent the same elements throughout the drawings.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness

The terms and words used in the following description and claims are not limited to the bibliographical meanings, but, are merely used by the inventor to enable a clear and consistent understanding of the disclosure. Accordingly, it should be apparent those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Reference throughout this specification to “an aspect”, “another aspect” or similar language means that a particular feature, structure, or characteristic described in connection with the embodiment is included in at least one embodiment of the disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skilled in the art to which this disclosure belongs. The system, methods, and examples provided herein are illustrative only and not intended to be limiting.

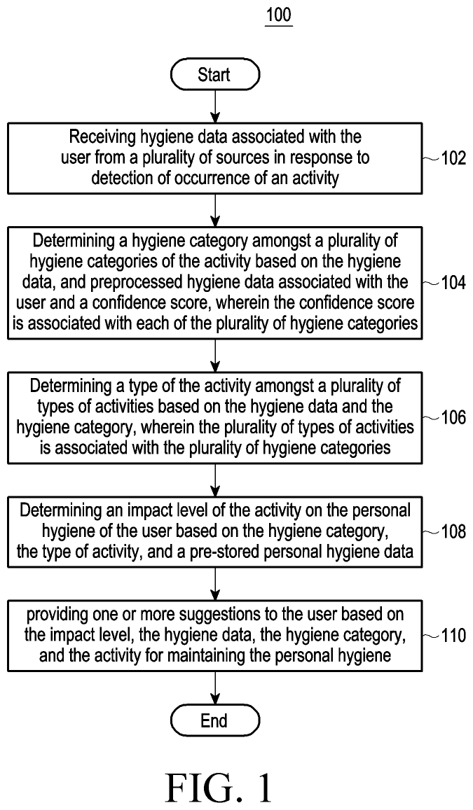

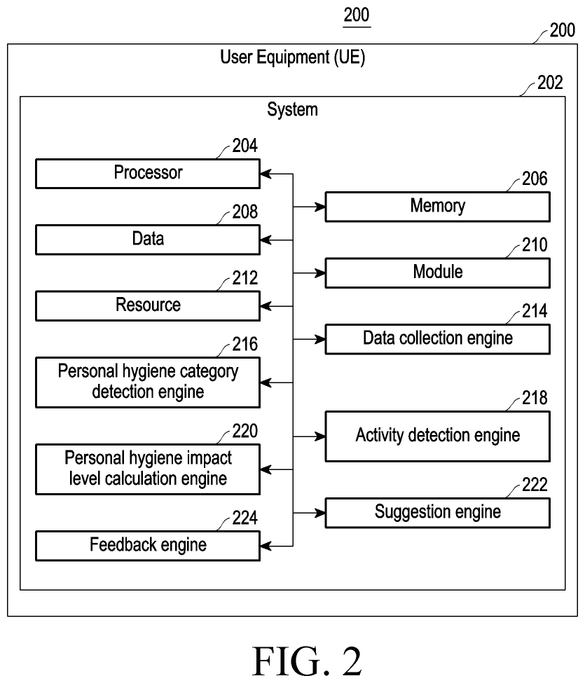

For the sake of clarity, the first digit of a reference numeral of each component of the disclosure is indicative of the Figure number, in which the corresponding component is shown. For example, reference numerals starting with digit “1” are shown at least in FIG. 1. Similarly, reference numerals starting with digit “2” are shown at least in FIG. 2, and so on and so forth.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a Wi-Fi chip, a Bluetooth® chip, a global positioning system (GPS) chip, a near field communication (NFC) chip, connectivity chips, a sensor controller, a touch controller, a finger-print sensor controller, a display drive integrated circuit (IC), an audio CODEC chip, a universal serial bus (USB) controller, a camera controller, an image processing IC, a microprocessor unit (MPU), a system on chip (SoC), an IC, or the like.

FIG. 1 illustrates a block diagram depicting a method 100 for providing one or more suggestions for maintaining personal hygiene of a user, according to an embodiment of the disclosure.

The method 100 may be implemented in an electronic device. Examples of the electronic device may include, but are not limited to, a smartphone, a laptop, a Personal Computer (PC), and a tablet. The method 100 may be configured to employ a Machine Learning (ML) technique to determine the one or more suggestions.

At block 102, the method 100 includes receiving hygiene data associated with the user from a plurality of sources in response to detection of occurrence of an activity.

At block 104, the method 100 includes determining a hygiene category amongst a plurality of hygiene categories of the activity based on the hygiene data, and preprocessed hygiene data associated with the user and a confidence score, wherein the confidence score is associated with each of the plurality of hygiene categories.

At block 106, the method 100 includes determining a type of the activity amongst a plurality of types of activities based on the hygiene data and the hygiene category, wherein the plurality of types of activities is associated with the plurality of hygiene categories.

At block 108, the method 100 includes determining an impact level of the activity on the personal hygiene of the user based on the hygiene category, the type of activity, and a pre-stored personal hygiene data.

At block 110, the method 100 includes providing one or more suggestions to the user based on the impact level, the hygiene data, the hygiene category, and the activity for maintaining the personal hygiene.

FIG. 2 illustrates a schematic block diagram 200 of a system 202 providing one or more suggestions for maintaining personal hygiene of a user, according to an embodiment of the disclosure.

In an embodiment, the system 202 may be incorporated in an electronic device. Examples of the electronic device may include, but are not limited to, a smartphone, a laptop, a PC, and a tablet. In an embodiment, the system 202 may be configured to provide one or more suggestion based on a hygiene data received from a number of sources. The system 202 may be configured to determine a hygiene category from a number of hygiene categories, a type of activity from a number of types of activities performed by the user, and an impact level related to the type of activity performed by the user for determining the one or more suggestions.

In one example embodiment, the system 202 can be a chip incorporated in the electronic device. In another example embodiment, the system 202 may be an implemented software, a logic-based program, a hardware, a configurable hardware, and the like. The system 202 includes a processor 204, memory 206, data 208, module(s) 210, resources(s) 212, a data collection engine 214, a personal hygiene category detection engine 216, an activity detection engine 218, a personal hygiene impact level calculation engine 220, a suggestion engine 222, and a feedback engine 224.

The processor 204, the memory 206, the data 208, the module(s) 210, the resources(s) 212, the data collection engine 214, the personal hygiene category detection engine 216, the activity detection engine 218, the personal hygiene impact level calculation engine 220, the suggestion engine 222, and the feedback engine 224 may be communicatively coupled to one another.

In an example, the processor 204 may be a single processing unit or a number of units, all of which could include multiple computing units. The processor 204 may be referred to as at least one processor. The processor 204 may be implemented as one or more microprocessors, microcomputers, microcontrollers, digital signal processors, central processing units, processor cores, multi-core processors, multiprocessors, state machines, logic circuitries, application-specific integrated circuits, field-programmable gate arrays and/or any devices that manipulate signals based on operational instructions. Among other capabilities, the processor 204 may be configured to fetch and/or execute computer-readable instructions and/or data stored in the memory 206.

In an example, the memory 206 may include any non-transitory computer-readable medium known in the art including, for example, volatile memory, such as static random-access memory (SRAM) and/or dynamic random-access memory (DRAM), and/or non-volatile memory, such as read-only memory (ROM), erasable programmable ROM (EPROM), flash memory, hard disks, optical disks, and/or magnetic tapes. The memory 206 may include the data 208.

The data 208 serves, amongst other things, as a repository for storing data processed, received, and generated by one or more of the processor 204, the module(s) 210, the resources(s) 212, the data collection engine 214, the personal hygiene category detection engine 216, the activity detection engine 218, the personal hygiene impact level calculation engine 220, the suggestion engine 222, and the feedback engine 224.

The module(s) 210, amongst other things, may include routines, programs, objects, components, data structures, etc., which perform particular tasks or implement data types. The module(s) 210 may also be implemented as, signal processor(s), state machine(s), logic circuitries, and/or any other device or component that manipulate signals based on operational instructions.

Further, the module(s) 210 may be implemented in hardware, instructions executed by at least one processing unit, for e.g., processor 204, or by a combination thereof. The processing unit may be a general-purpose processor which executes instructions to cause the general-purpose processor to perform operations or, the processing unit may be dedicated to performing the required functions. In another aspect of the disclosure, the module(s) 210 may be machine-readable instructions (software) which, when executed by a processor/processing unit, may perform any of the described functionalities.

In some example embodiments, the module(s) 210 may be machine-readable instructions (software) which, when executed by a processor/processing unit, perform any of the described functionalities.

The resource(s) 212 may be physical and/or virtual components of the system 202 that provide inherent capabilities and/or contribute towards the performance of the system 202. Examples of the resource(s) 212 may include, but are not limited to, memory (e.g., the memory 206), a power unit (example, a battery), a display unit, etc. The resource(s) 212 may include a power unit/battery unit, a network unit, etc., in addition to the processor 204, and the memory 206.

Continuing with the above embodiment, the data collection engine 214 may be configured to receive hygiene data related to the user from a number of sources. Furthermore, the number of sources may include at least one of a number of sensors and the memory 206 of the electronic device. Examples of the number of sensors may include, but are not limited to, an accelerometer, a gyroscope, a temperature sensor, a Global Positioning System (GPS) sensor, an Electrocardiogram (ECG) sensor, an ambient light sensor, a barometer, an Electrodermal Activity (EDA) sensor, Bioelectrical Impedance Analysis (BIA) sensor. In an embodiment, the number of sensors may be in vicinity of the user. The number of sensors may be present in the electronic device of the user. In another embodiment, each sensor from the number of sensors may be a wearable sensor worn by the user.

Moving forward, the data collection engine 214 may be configured to receive the hygiene data in response to detection of occurrence of an activity. The activity may be performed by the user and detected by at least one sensor from the number of sensors. The at least one sensor may be communicating with the system 202 and further may be configured to notify the system 202 about the occurrence of the activity. Further, the hygiene data received from the number of sources may include at least one of sensor data received from at least one sensor amongst the number of sensors, user data related to one or more of a reminder and an alarm for performing one or more of the number of types of activities set up by the user in the electronic device, environmental data including at least of a temperature, a humidity, and precipitation, associated with a vicinity of the user, pre-stored personal hygiene data associated with the user, routine data generated by analyzing a daily routine of the user, location associated with the user at a time of occurrence of the activity, contextual data generated from a combination of at least two of the sensor data, the user data, the environmental data, the pre-stored personal hygiene data, and the routine data.

Continuing with the above embodiment, the sensors data may be related to a movement of a body part of the user. In an embodiment, the user may be moving an arm to perform an activity such as smoking, and brushing teeth. The user data indicate that the user performs one of the types of activities at a particular time in a day. In an embodiment, the particular type of activity may be one or more of drinking water, bathing, washing hair, brushing teeth, and flossing teeth. The pre-stored personal hygiene data information related to the personal hygiene stored by the user in the memory 206. Examples of the pre-stored personal hygiene data may include information related to products used by the user for skin and hair care, or the like. Further, the routine data may be generated based on processing of the user data, and sensor data for a pre-determined period of time by a daily routine analyzer incorporated within the data collection engine 214.

Subsequent to reception of the hygiene data by the data collection engine 214, the personal hygiene category detection engine 216 may be configured to determine hygiene category from the number of hygiene categories of the activity performed by the user. Examples of the number of hygiene categories may include, but are not limited to, an oral hygiene category, a skin hygiene category, a hair hygiene category, a sanitization hygiene category, and a combination of at least two of the oral hygiene category, the skin hygiene category, the hair hygiene category, and the sanitization hygiene category. The hygiene category may be detected based on the hygiene data, preprocessed hygiene data associated with the user. The preprocessed hygiene data may include information associated with ideal hygiene data for the user. For determining the hygiene category, the personal hygiene category detection engine 216 may be configured to employ a number of hygiene engines incorporated within the personal hygiene category detection engine 216. Each of the number of hygiene engines may be related to a hygiene category amongst the number of hygiene categories.

Continuing with the above embodiment, each hygiene engine may be configured to determine a similarity score between the hygiene data and the preprocessed hygiene data. The similarity score may indicate a similarity between the hygiene data and the preprocessed hygiene data. Moving forward from determining the similarity score, each hygiene engine may be configured to generate a weight-hygiene pair for the respective hygiene category. The weight-hygiene pair may be generated by pairing the similarity score with a weight associated with the respective hygiene category amongst the number of hygiene categories. Subsequent to generating the weight-hygiene pair, each hygiene engine may be configured to calculate a confidence score related to a corresponding hygiene category. The confidence score may be calculated based on each weight-hygiene pair generated by each hygiene engine from the number of hygiene engines. Further, the personal hygiene category detection engine 216 may be configured to compare the confidence score related to each hygiene category and select the hygiene category with a highest confidence score.

To that understanding, in response to the determination of the hygiene category by the personal hygiene category detection engine 216, the activity detection engine 218 may be configured to determine a type of the activity from the number of types of activities. The type of the activity may be determined based on the hygiene data and the hygiene category. The number of types of activities may be related to the number of hygiene categories. Examples of the number of types of hygiene activities may include, but are not limited to, brushing teeth, smoking, flossing, sanitizing, washing hands, drinking, eating, rinsing mouth, bathing, receiving sun exposure, sweating, going out in a number of weather conditions, wearing a headgear, removing a headgear, sneezing, coughing, performing a number of hand activities, and washing hair. For determining the type of activity, the activity detection engine 218 may be configured to employ a ML technique. The activity detection engine 218 may be configured to employ the ML technique to correlate the hygiene data, the hygiene category, the pre-processed hygiene data with one another for determining the type of the hygiene activity.

Continuing with the above embodiment, the personal hygiene impact level calculation engine 220 may be configured to determine an impact level of the activity on the personal hygiene of the user. The impact level may be determined based on the hygiene category, the type of activity, and a pre-stored personal hygiene data. For determining the impact level, the personal hygiene impact level calculation engine 220 may be configured to assign an activity weight to the activity and a duration weight to a time duration of the activity. Further, the personal hygiene impact level calculation engine 220 may be configured to calculate an activity score based on the activity weight, the duration weight, a previous activity score associated with a previous activity, and a weight associated with the previous activity score. the weight associated with the previous activity score may be based on a time duration between occurrence of the previous activity and the activity. Moving forward, the personal hygiene impact level calculation engine 220 may be configured to compare the activity score with the previous activity score to determine the impact level. The impact level may be one of a low impact level, a medium impact level, and a high impact level.

Subsequent to determination of the impact level by the personal hygiene impact level calculation engine 220, the suggestion engine 222 may be configured to provide one or more suggestions to the user. The one or more suggestions may be generated based on the impact level, the hygiene data, the hygiene category, and the activity for maintaining the personal hygiene. Examples of the one or more suggestions may include, but are not limited to, brushing teeth, using a mouthwash, using a mouth freshener, rinsing mouth with water, drinking water, washing hands, taking a bath, washing hair, applying hair products to hair, combing hair, changing clothes, using a towel to dry one or more body parts, applying skincare products, using an umbrella, sanitizing hands, wearing a face mask, and wearing gloves.

For providing the one or more suggestions, the suggestion engine 222 may be configured to identify one or more possible suggestions amongst a number of pre-stored possible suggestions in a database. The one or more possible suggestions may be identified based on the hygiene data, the hygiene category, the type of activity, and the impact level. The number of pre-stored possible suggestions may be mapped with the number of hygiene categories and the number of types of activities in the database. The database may be stored in the memory 206.

Further, the suggestion engine 222 may be configured to identify the one or more suggestions from the one or more possible suggestions. The one or more suggestions may be identified based on the hygiene data, the impact level, one or more previous suggestions suggested to the user, and feedback associated with usage of one or more previous suggestions suggested to the user. The one or more suggestions may be provided to the user on an interface of the electronic device.

Continuing with the above embodiment, the feedback engine 224 may be configured to determine whether the one or more suggestions is performed by the user to maintain the personal hygiene. Furthermore, the feedback engine 224 may be configured to generated feedback based on the determination.

The operations of the engines (e.g. data collection engine 214, personal hygiene category detection engine 216, personal hygiene category detection engine 218, personal hygiene impact level calculation engine 220, suggestion engine 222, and feedback engine 224) may be performed by at least one processor (i.e. the processor 204) executing instructions stored in a non-transitory computer readable storage medium (i.e. the memory 206).

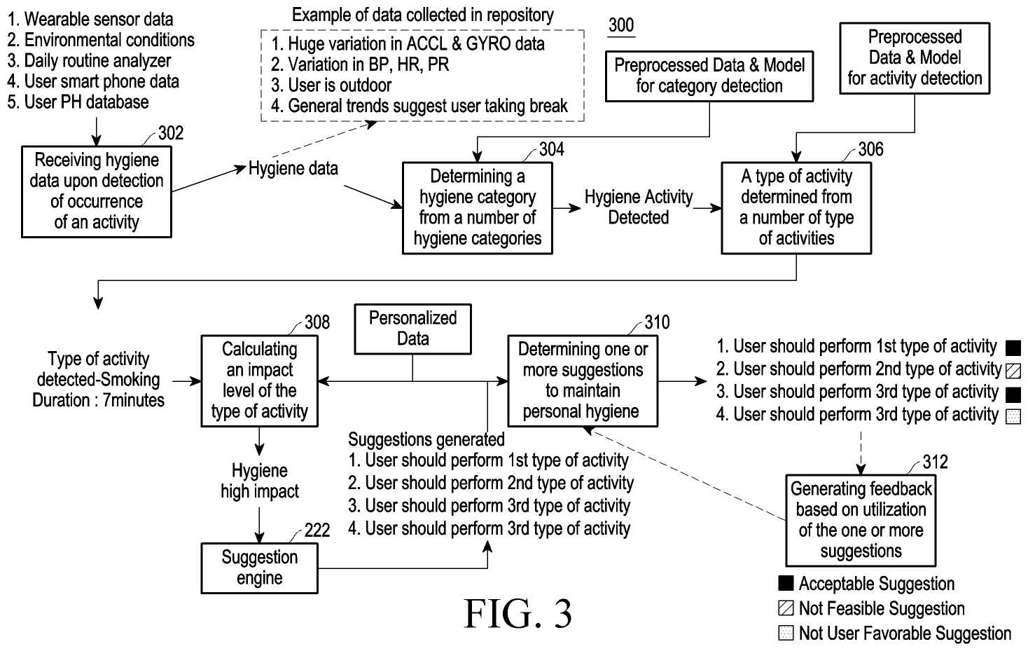

FIG. 3 illustrates an operational flow diagram depicting a process 300 for providing one or more suggestions for maintaining personal hygiene of a user, according to an embodiment of the disclosure. The process may be performed by the system 202 incorporated in an electronic device of the user.

At operation 302, the process 300 may include receiving hygiene data related to the user from a number of sources. The number of sources may be at least one the memory 206 and a number of sensors. The hygiene data may be received by the data collection engine 214 as referred in the FIG. 2 in response to detection of occurrence of activity. The activity may be detected by one or more of the number of sensors and performed by the user. Examples of the number of sensors may include, but are not limited to, an accelerometer, a gyroscope, a temperature sensor, a GPS sensor, an ECG sensor, an ambient light sensor, a barometer, an EDA sensor, and a BIA sensor. In an embodiment, the number of sensors may be in vicinity of the user. The number of sensors may be present in the electronic device of the user. In another embodiment, each sensor from the number of sensors may be a wearable sensor worn by the user.

Continuing with the above embodiment, the hygiene data may be a collection of various types of data generated and share by the number of sources. Further, the hygiene data may include at least one of sensor data received from at least one sensor amongst the number of sensors, user data related to one or more of a reminder and an alarm for performing one or more of the number of types of activities set up by the user in the electronic device, environmental data including at least of a temperature, a humidity, and precipitation, associated with a vicinity of the user, pre-stored personal hygiene data associated with the user, routine data generated by analyzing a daily routine of the user, location associated with the user at a time of occurrence of the activity, contextual data generated from a combination of at least two of the sensor data, the user data, the environmental data, the pre-stored personal hygiene data, and the routine data.

Continuing with the above embodiment, the sensors data may be related to a movement of a body part of the user. In an embodiment, the user may be moving an arm to perform an activity such as smoking, and brushing teeth. The user data indicate that the user performs one of the types of activities at a particular time in a day. In an embodiment, the particular type of activity may be one or more of drinking water, bathing, washing hair, brushing teeth, and flossing teeth. The pre-stored personal hygiene data information related to the personal hygiene stored by the user in the memory 206. Examples of the pre-stored personal hygiene data may include information related to products used by the user for skin and hair care, or the like. Further, the routine data may be generated based on processing of the user data, and sensor data for a pre-determined period of time by a daily routine analyzer incorporated within the data collection engine 214.

At operation 304, the process 300 may include determining a hygiene category from a number of hygiene categories of the activity. The hygiene category may be determined by the personal hygiene category detection engine 216 as referred in the FIG. 2. Examples of the number of hygiene categories may include, but are not limited to, an oral hygiene category, a skin hygiene category, a hair hygiene category, a sanitization hygiene category, and a combination of at least two of the oral hygiene category, the skin hygiene category, the hair hygiene category, and the sanitization hygiene category. The hygiene category may be determined based on the hygiene data, preprocessed hygiene data associated with the user. The preprocessed hygiene data may include information associated with ideal hygiene data for the user. The process 300 may include utilizing a number of hygiene engines incorporated within the personal hygiene category detection engine 216 to determine the hygiene category. Each of the number of hygiene engines may be related to a hygiene category amongst the number of hygiene categories.

Continuing with the above embodiment, each hygiene engine may be configured to determine a similarity score between the hygiene data and the preprocessed hygiene data indicating a similarity between the hygiene data and the preprocessed hygiene data. Further, the process 300 may include pairing the similarity score with a weight associated with the respective hygiene category associated with each hygiene engine to generate a weight-hygiene pair. Further, the process 300 may include calculating by each hygiene engine a confidence score related to a corresponding hygiene category. The confidence score may be calculated based on each weight-hygiene pair generated by each hygiene engine. Further, the process 300 may include comparing by the personal hygiene category detection engine 216, the confidence score related to each hygiene category and select the hygiene category with a highest confidence score.

At operation 306, the process 300 may include determining a type of the activity from a number of types of activities. The type of activity may be determined by the activity engine as referred in the FIG. 2 based on the hygiene data and the hygiene category. Each type of activity may be a subset of at least one of the number of hygiene categories. Examples of the number of types of hygiene activities may include, but are not limited to, brushing teeth, smoking, flossing, sanitizing, washing hands, drinking, eating, rinsing mouth, bathing, receiving sun exposure, sweating, going out in a number of weather conditions, wearing a headgear, removing a headgear, sneezing, coughing, performing a number of hand activities, and washing hair. The process 300 may include employ a ML technique to correlate the hygiene data, the hygiene category, the pre-processed hygiene data with one another for determining the type of the hygiene activity.

At operation 308, the process 300 may include determining an impact level of the activity on the personal hygiene of the user by the personal hygiene impact level calculation engine 220 as referred in the FIG. 2. Determining the impact level may include assigning an activity weight to the activity and a duration weight to a time duration of the activity. Further, an activity score may be calculated based on the activity weight, the duration weight, a previous activity score associated with a previous activity, and a weight associated with the previous activity score. The weight associated with the previous activity score may be based on a time duration between occurrence of the previous activity and the activity. Moving forward, the process 300 may include comparing the activity score with the previous activity score to determine the impact level. The impact level may be one of a low impact level, a medium impact level, and a high impact level.

At operation 310, the process 300 may include providing one or more suggestions to the user. The one or more suggestions may main the personal hygiene of the user. The one or more suggestions may be generated by the suggestion engine 222 as referred in the FIG. 2. Examples of the one or more suggestions may include, but are not limited to, brushing teeth, using a mouthwash, using a mouth freshener, rinsing mouth with water, drinking water, washing hands, taking a bath, washing hair, applying hair products to hair, combing hair, changing clothes, using a towel to dry one or more body parts, applying skincare products, using an umbrella, sanitizing hands, wearing a face mask, and wearing gloves.

Continuing with the above embodiment, for providing the one or more suggestions, the process 300 may include identifying one or more possible suggestions amongst a number of pre-stored possible suggestions in a database based on the hygiene data, the hygiene category, the type of activity, and the impact level. The number of pre-stored possible suggestions may be mapped with the number of hygiene categories and the number of types of activities in the database stored in the memory 206. Furthermore, the process 300 may include identifying the one or more suggestions from the one or more possible suggestions based on the hygiene data, the impact level, one or more previous suggestions suggested to the user, and feedback associated with usage of one or more previous suggestions suggested to the user. The one or more suggestions may be provided to the user on an interface of the electronic device.

At operation 312, the process 300 may include determining by the feedback engine 224 whether the one or more suggestions is performed by the user to maintain the personal hygiene. Furthermore, the feedback engine 224 may be configured to generate feedback based on the determination.

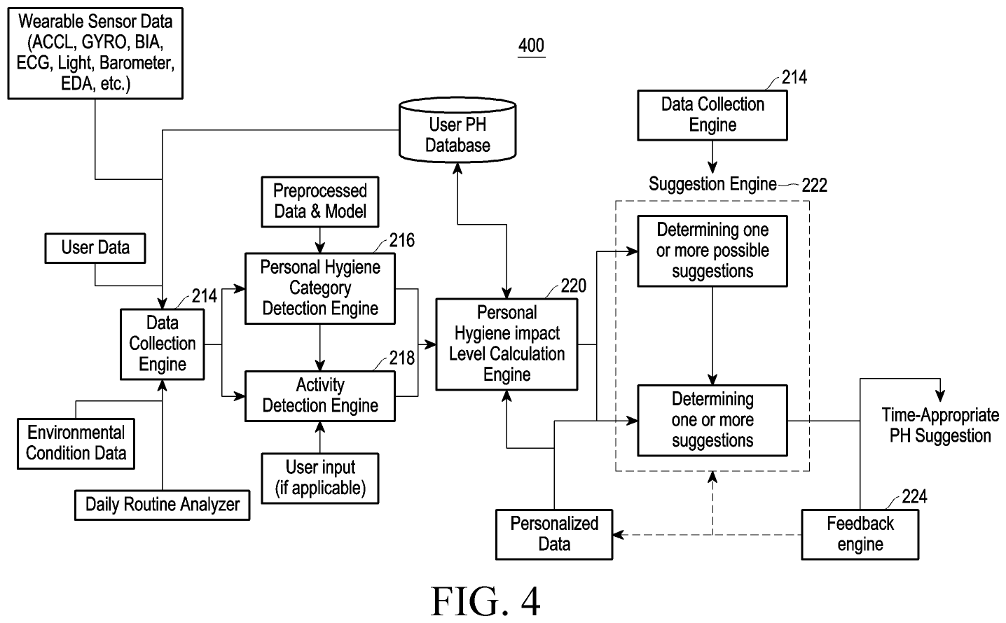

FIG. 4 illustrates an architectural diagram depicting a method 400 for providing one or more suggestions for maintaining personal hygiene of a user, according to an embodiment of the disclosure. The one or more suggestions may be determined based on occurrence of an activity. The activity may be performed by the user.

The data collection engine 214 may be configured to collect sensor data, user data, environmental data, and personal hygiene data from a database in the memory 206 trained and processed with a daily routine analyzer to generate hygiene data. The hygiene data may be utilized by the personal hygiene category detection engine 216, and the activity detection engine 218.

The personal hygiene category detection engine 216 may be configured to receive the hygiene data feed the hygiene data to a number of hygiene engines. The number of hygiene engines may analyze a variation in the hygiene data by utilizing preprocessed hygiene data associated with the user. Further, the personal hygiene category detection engine 216 may calculate a confidence level from the hygiene data. A hygiene category amongst a number of hygiene categories above a certain threshold may be considered and processed further by the activity detection engine 218.

The activity detection engine 218 may be configured to receive an input from the data collection engine 214 and the personal hygiene category detection engine 216 to detect and select a specific a type of activity that may impact a personal hygiene level of a user.

Further, the personal hygiene impact level calculation engine 220 may be configured to receive an input from each of the data collection engine 214, the personal hygiene category detection engine 216, and the activity detection engine 218. Further, the personal hygiene impact level calculation engine may utilize a past personal hygiene history of the user stored in the database, personalized user data, and identified types of activities and a duration of the types of activities to determine an impact on an identified hygiene activity and the personal hygiene level of the user and storing data associated with the impact level in the database.

Further, the suggestion engine 222 receives in an input from each of the data collection engine 214 and the personal hygiene impact level calculation engine 220 a past personal hygiene history of the user, and identified types of activities and the impact level to generate one or more possible suggestions. Further, the suggestion engine 222 may be configured to check a feasibility of the one or more possible suggestions as per data history of the user, a current condition, a schedule, and the like. Based on that, the one or more suggestions may be generated for the user such that the one or more suggestions may be time appropriate.

Further, the feedback engine 224 may track the user to check if the one or more suggestions is performed by the user or not to enhance an over-all system for future usage. Further, feedback may be provided to the suggestion engine 222.

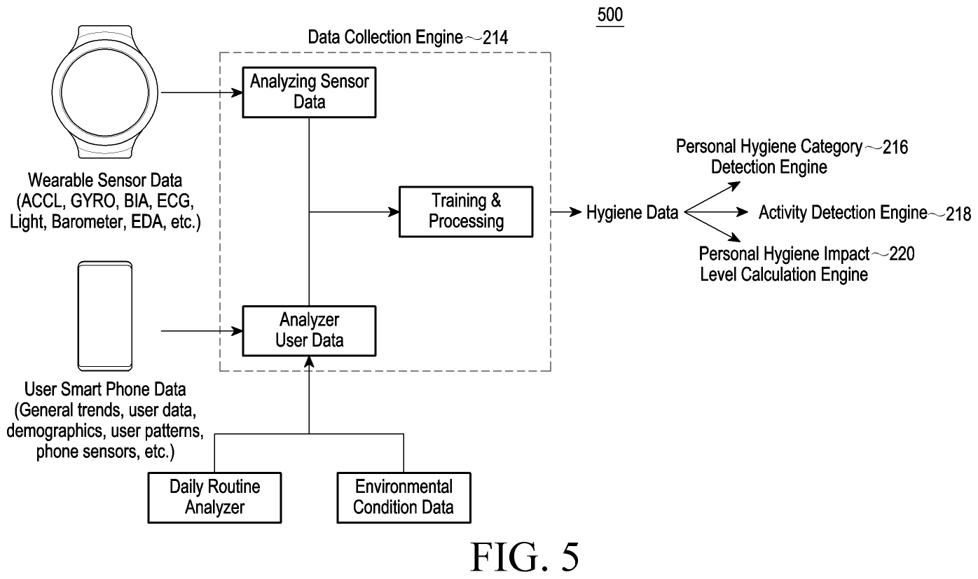

FIG. 5 illustrates an operational flow diagram depicting a process 500 to receive hygiene data, according to an embodiment of the disclosure.

The process 500 may be performed by the data collection engine 214 as referred in the FIG. 2. The hygiene data may be received from a number of sources. The number of sources may include a number of sensors and the memory 206 as referred in the FIG. 2. The number of sensors may include an accelerometer, a gyroscope, a temperature sensor, a GPS sensor, an ECG sensor, an ambient light sensor, a barometer, an EDA sensor, and a BIA sensor and the like. In an embodiment, the number of sensors may be incorporated within an electronic device of a user. In an embodiment, the number of sensors may be wearable sensors worn by the user. In another embodiment, the number of sensors may be places in vicinity of the user.

Continuing with the above embodiment, the data collection engine 214 may be configured to receive the hygiene data in response occurrence of an activity by the user. The number of sensors may detect the occurrence of the activity and communicate the occurrence to the system 202 incorporating the data collection engine 214. Further, the hygiene data received from the number of sources may include at least one of sensor data received from at least one sensor amongst the number of sensors, user data related to one or more of a reminder and an alarm for performing one or more of the number of types of activities set up by the user in the electronic device, environmental data including at least of a temperature, a humidity, and precipitation, associated with a vicinity of the user, pre-stored personal hygiene data associated with the user, routine data generated by analyzing a daily routine of the user, location associated with the user at a time of occurrence of the activity, contextual data generated from a combination of at least two of the sensor data, the user data, the environmental data, the pre-stored personal hygiene data, and the routine data.

Continuing with the above embodiment, the sensor data may be related to a movement of a body part of the user. In an embodiment, the user may be moving an arm to perform an activity such as smoking, and brushing teeth. The user data indicate that the user performs one of the types of activities at a particular time in a day. In an embodiment, the particular type of activity may be one or more of drinking water, bathing, washing hair, brushing teeth, and flossing teeth. The pre-stored personal hygiene data information related to the personal hygiene stored by the user in the memory 206. Examples of the pre-stored personal hygiene data may include information related to products used by the user for skin and hair care, or the like. Further, the routine data may be generated based on processing of the user data, and sensor data for a pre-determined period of time by a daily routine analyzer incorporated within the data collection engine 214.

The user collection engine may be configured to receive and process the at least one of sensor data, the user data, the environmental data, the pre-stored personal hygiene data, the routine data, the location, the contextual data to generate the hygiene data.

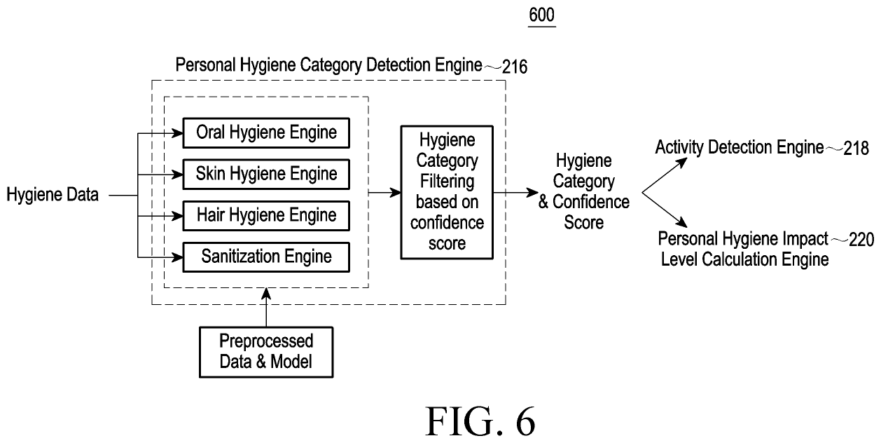

FIG. 6 illustrates an operational flow diagram depicting a process 600 for detecting a hygiene category related to an activity performed by a user, according to an embodiment of the disclosure.

The hygiene category may be determined from a number of hygiene categories by the personal hygiene category detection engine 216 as referred in the FIG. 2. Examples of the number of hygiene categories may include, but are not limited to, an oral hygiene category, a skin hygiene category, a hair hygiene category, a sanitization hygiene category, and a combination of at least two of the oral hygiene category, the skin hygiene category, the hair hygiene category, and the sanitization hygiene category. The hygiene category may be detected based on the hygiene data, preprocessed hygiene data associated with the user. The preprocessed hygiene data may include information associated with ideal hygiene data for the user. The hygiene data and the preprocessed hygiene data may be transmitted to a number of hygiene engines in the personal hygiene category detection engine 216 and each engine may be related to a hygiene category. Each hygiene engine may be configured to analyze a variation in data category related to the hygiene category associated with the respective hygiene engine. Based on the variation, a confidence score may be calculated to determine whether the hygiene category is to be marked as detected.

The confidence score may be calculated based on a similarity between the hygiene data and the preprocesses hygiene data. The similarity may be determined based on the variation analyzed by each hygiene engine. The similarity determined at each hygiene engine may correspond to a similarity score and the similarity score may be paired with a weight related to the respective hygiene category amongst the number of hygiene categories to generate a weight-hygiene pair for the respective hygiene category. Further, a summation of each hygiene-weight pair for each hygiene category may be performed to generate the confidence score. The confidence score H_{C} may be calculated as depicted in the below mentioned Equation 1:

$$\begin{matrix}{H}_{C} = {S}_{A} * {W}_{A} + {S}_{G} * {W}_{G} ++ {S}_{AL} * {W}_{AL} + {S}_{HR} * {W}_{HR} , \\ {H}_{C} varies between 0 and 1 \\ {W}_{A} + {W}_{G} ++ {W}_{AL} + {W}_{HR} = 1\end{matrix} \qquad Equation 1$$

wherein, S_{A} may indicate the similarity in accelerometer data, S_{G} may indicate the similarity in gyroscope data, S_{AL} may indicate the similarity in ambient light data, S_{HR} may indicate the similarity in heart rate data, WA may indicate a weight of the similarity the accelerometer data, W_{G} may indicate a weight of the similarity in the gyroscope data, W_{AL} may indicate a weight of the similarity the ambient light data, and W_{HR} may indicate a weight of the similarity in the heart rate data.

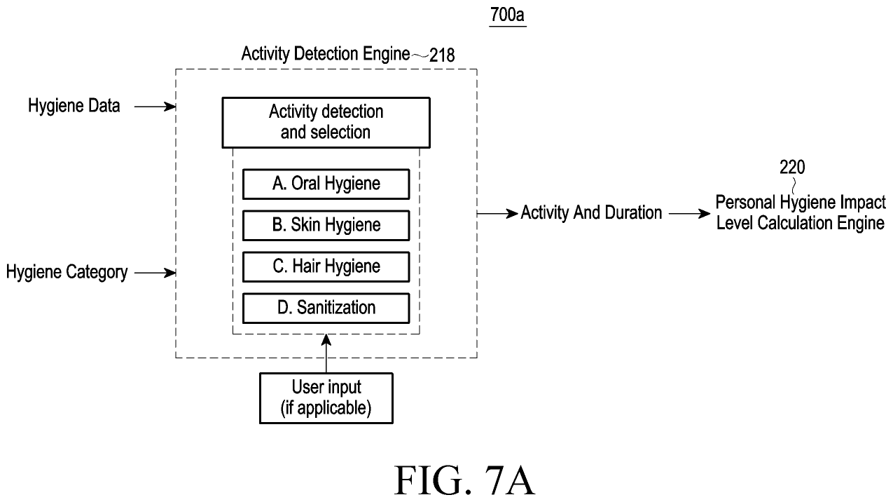

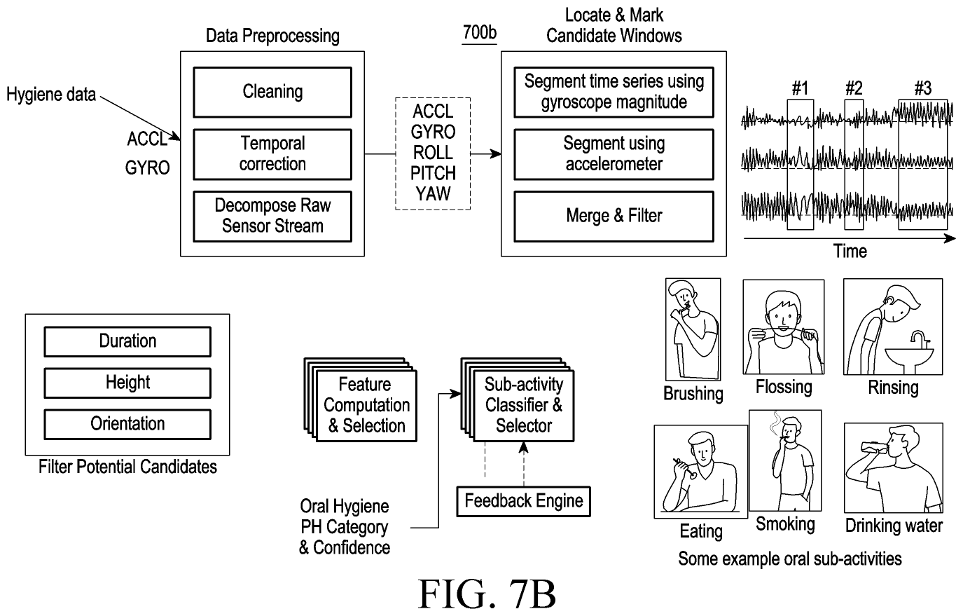

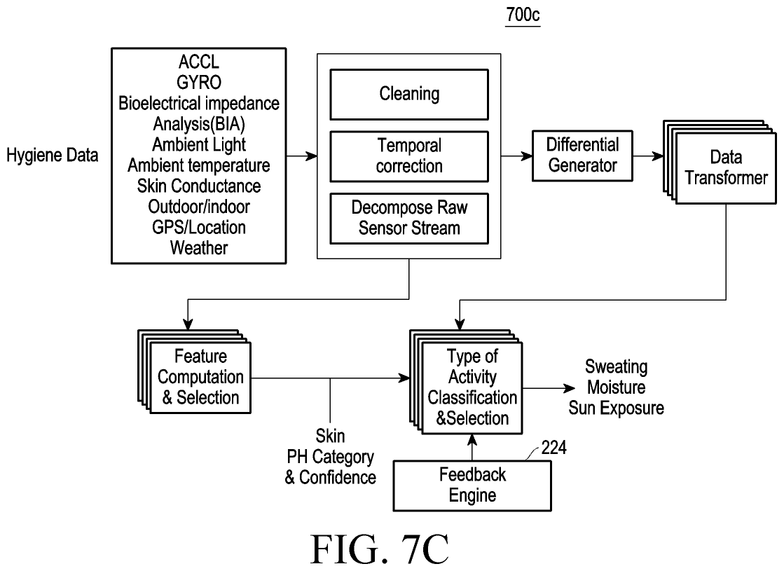

FIG. 7A illustrates an operational flow diagram depicting a process 700a for determining a type of hygiene activity performed by a user, according to an embodiment of the disclosure.

The type of hygiene activity may be determined from a number of types of hygiene activities by the activity detection engine 218 as referred in the FIG. 2. The type of the hygiene activity may be determined based on hygiene data related to the user and a hygiene category. The number of types of hygiene activities may be related to the number of hygiene categories. Examples of the number of types of hygiene activities may include, but are not limited to, brushing teeth, smoking, flossing, sanitizing, washing hands, drinking, eating, rinsing mouth, bathing, receiving sun exposure, sweating, going out in a number of weather conditions, wearing a headgear, removing a headgear, sneezing, coughing, performing a number of hand activities, and washing hair. For determining the type of activity, the activity detection engine 218 may be configured to employ a ML technique to correlate the hygiene data, the hygiene category, the pre-processed hygiene data with one another for determining the type of the hygiene activity.

FIG. 7B illustrates an operational flow diagram depicting a process 700b for determining a type of activity associated with an oral hygiene category, according to an embodiment of the disclosure. The type of activity associated with the oral hygiene category may be one of brushing, flossing, rinsing, smoking, eating, and

drinking. The activity detection engine 218 may utilize sensor data generated by a number of sensors such as an accelerometer, and a gyroscope, and one or more bio-sensors, a GPS sensor, an ECG sensor, an ambient light sensor, a barometer, an EDA sensor, and a BIA sensor to determine the type of activity accurately. The activity detection engine 218 may also utilize feedback generated by the feedback engine 224 as referred in the FIG. 2. The sensor data may indicate one or more hand to mouth gestures performed by the user.

FIG. 7C illustrates an operational flow diagram depicting a process 700c for determining a type of activity associated with a skin hygiene category, according to an embodiment of the disclosure. A hygiene level related to skin may be based on at least one of the number of the type of activities such as bathing,

sweating, going out in a number of weather conditions, exposing skin to direct sunlight. The activity detection engine 218 may be configured to receive the hygiene data such as sensor data from the number of sensors, environmental data including at least of a temperature, a humidity, and precipitation, associated with a vicinity of the user, pre-stored personal hygiene data associated with the user such as information related to a skin conductance of the user, and location associated with the user at a time of occurrence of an activity. In an embodiment, the activity detection engine 218 may be configured to perform a Bioelectrical Impedance Analysis (BIA) on the hygiene data to determine the type of activity. Further, based on a variation in a light intensity with respect to an indoor location, an outdoor location, and a semi-outdoor location combined with an ambient temperature and a weather forecast at the location of the user may provide an accuracy in determining the type of activity.

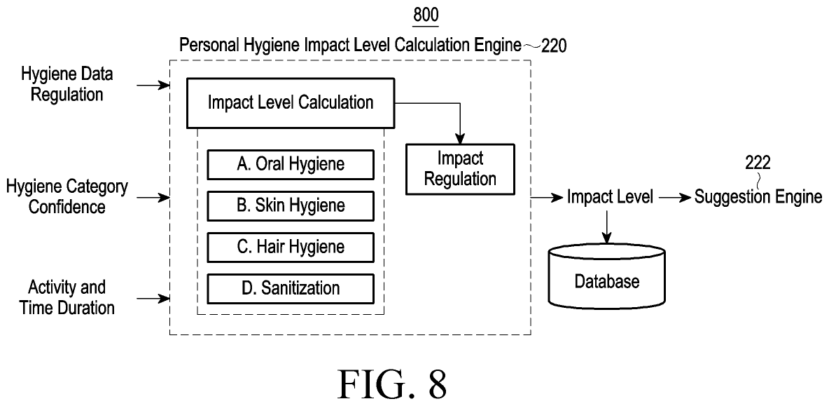

FIG. 8 illustrates an operational flow diagram depicting a process 800 for determining an impact level of an activity on a personal hygiene of the user, according to an embodiment of the disclosure.

The impact level may indicate an impact of the activity on the personal hygiene of the user. The impact may be one of a positive impact, and a negative impact. The activity may be performed by the user. The impact level may be determined based on a hygiene category of the hygiene activity, a type of the hygiene activity, and a pre-stored personal hygiene data by the personal hygiene impact level calculation engine 220 as referred in the FIG. 2. The personal hygiene impact level calculation engine 220 may also utilize a time duration related to the occurrence of the activity, a time duration between a previous activity and the activity performed by the user, and a previous activity score of the previous activity.

Continuing with the above embodiment, the personal hygiene impact level calculation engine 220 may be configured to observe a change in an activity score with respect to a previous activity score to determine the impact level. The activity score may be dynamical in nature as for similar conditions, weights related to a number of type of activities may be different based on user data and a past feedback.

Continuing with the above embodiment, the personal hygiene impact level calculation engine 220 may be configured to assign an activity weight to the activity and a duration weight to a time duration of the activity. Further, the personal hygiene impact level calculation engine 220 may be configured to calculate the activity score based on the activity weight, the duration weight, the previous activity score associated with the previous activity, and a weight associated with the previous activity score. The weight associated with the previous activity score may be based on a time duration between occurrence of the previous activity and the activity. Moving forward, the personal hygiene impact level calculation engine 220 may be configured to determine the impact level by comparing the activity score with the previous activity score. The personal hygiene impact level calculation engine 220 may be configured to monitor the activity score of a hygiene category on a scale. In an embodiment, the scale may range between values 0 through 20.

Further, the impact level may be categorized in a number of levels. The number of levels may include a low level, a medium level, and a high level. The low value may correspond to a change in the activity score with respect to the previous activity between value 0.1 to 1.5. The medium value may correspond to the change in the activity score with respect to the previous activity between value 1.5 to 3. The high value may correspond to the change in the activity score with respect to the previous activity for a value greater than 3.

The activity score (Oi (Ai, ti)) may be calculated based on the below mentioned Equation 2:

$$\begin{matrix}{O}_{i} ( {A}_{i} , {t}_{i} ) = {O}_{i - 1} ( {A}_{i - 1} , {t}_{i - 1} ) * {W}_{Oi - 1} + {W}_{Ai} * {W}_{Ti} . \\ wherein , {O}_{i - 1} ( {A}_{i - 1} , {t}_{i - 1} ) -> previous activity score , \\ {W}_{Oi - 1} -> weight of the previous activity score based on a time lapsed between the previous activity and the activity , {W}_{A 1} -> the weight of the activity , and {W}_{T 1} -> weight of the duration for the activity .\end{matrix} \qquad Equation 2$$

Further, the impact level may be calculated based on the change in the activity score and the previous activity score.

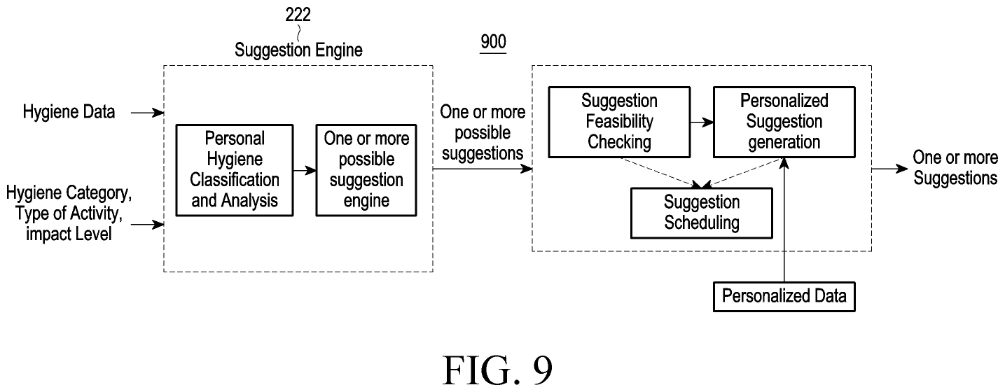

FIG. 9 illustrates an operational flow diagram depicting a process 900 for determining one or more suggestions, according to an embodiment of the disclosure.

The one or more suggestions may be determined and provided to a user by the suggestion engine 222 as referred in the FIG. 2. The one or more suggestions may be provided to the user in response to occurrence of an activity that may impact a hygiene category related to the activity. The activity may be performed by the user. Examples of the one or more suggestions may include, but are not limited to, brushing teeth, using a mouthwash, using a mouth freshener, rinsing mouth with water, drinking water, washing hands, taking a bath, washing hair, applying hair products to hair, combing hair, changing clothes, using a towel to dry one or more body parts, applying skincare products, using an umbrella, sanitizing hands, wearing a face mask, and wearing gloves.

Continuing with the above embodiment, the suggestions engine may be configured to identify one or more possible suggestions from a number of pre-stored possible suggestions. The number of pre-store possible suggestions may be stored in a database in the memory 206. Further, the one or more possible suggestions may be identified based on hygiene data, a hygiene category, a type of activity, an impact level related to the user. For each hygiene category a number of hygiene categories, and each type of hygiene activity from a number of type of activities, the number of pre-stored suggestions may be mapped to a number of impact levels varying one a user context as depicted in Table 1. The number of impact levels may be amongst a low level, a medium level, and a high level. In an embodiment, an oral hygiene category may include cleansing of a mouth to eliminate one or more of a mouth odor, and a buildup of bacteria. For example, Oral Hygiene suggestions mainly comprise of cleansing the mouth to eliminate mouth odor or buildup of bacteria.

TABLE 1

| Type of Activity | Impact Level | One or more possible suggestions |
|---|---|---|
| Smoking | HIGH | 1. User should rinse mouth |
| | | 2. User should brush teeth |
| | | 3. User should wash hands |
| | | 4. User should use Listerine |
| Smoking | MEDIUM | 1. User should wash hands |
| | | 2. User should rinse mouth |
| Smoking | LOW | 1. User should eat mouth freshener |
| | | 2. User should wash hands |
| Eating | HIGH | 1. User should brush teeth |
| | | 2. User should rinse mouth |
| | | 3. User should drink water |
| Eating | MEDIUM | 1. User should rinse mouth |
| | | 2. User should drink water |
| Eating | LOW | 1. User should drink water |
| Passive Time Flow | HIGH | 1. User should rinse mouth |
| | | 2. User should drink water |
| | | 3. User should brush teeth |
| | | 4. User should use Listerine |
| Passive Time Flow | MEDIUM | 1. User should rinse mouth |
| | | 2. User should drink water |
| Passive Time Flow | LOW | 1. User should drink water |

Table 1 depicts the type of activity, the impact level, and the one or more possible suggestions.

Continuing with the above embodiment, the suggestion engine 222 may be configured to identify the one or more suggestions from the one or more possible suggestions. The one or more suggestions may be identified based on the hygiene data, the impact level, one or more previous suggestions suggested to the user, and feedback associated with usage of one or more previous suggestions suggested to the user. The suggestions engine may be configured to check a feasibility of the one or more possible suggestions based on the user context. Upon checking, the suggestion engine 222 may be configured to personalize the one or more possible suggestions and identify the one or more suggestions.

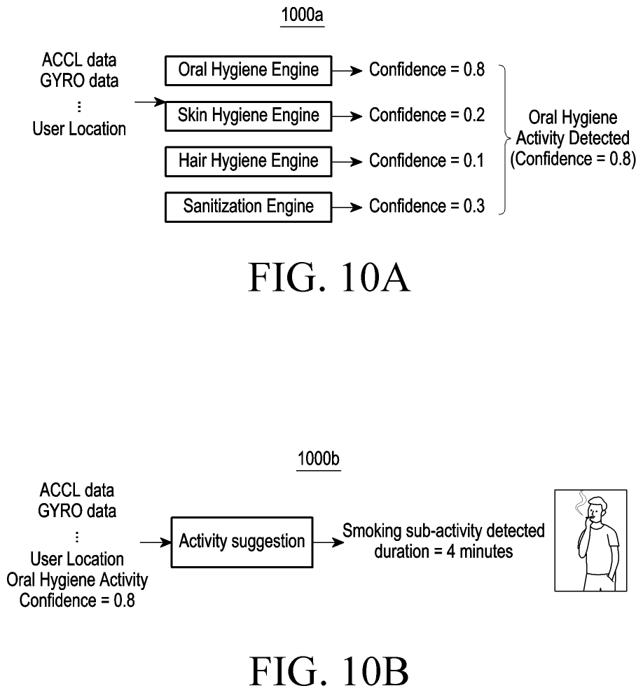

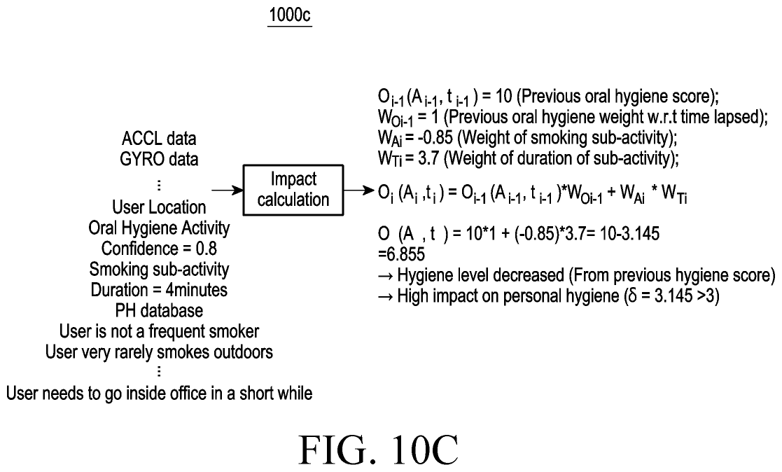

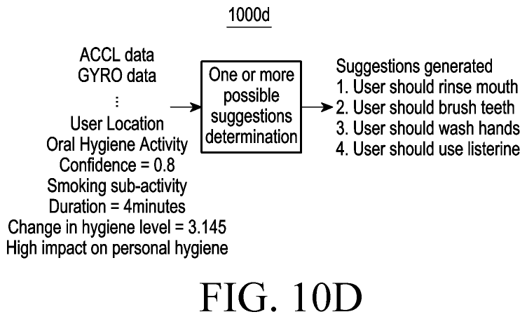

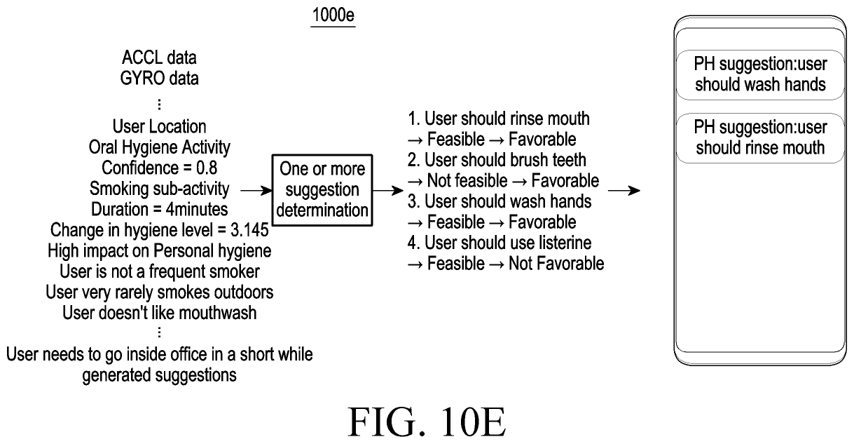

FIG. 10A illustrates a use case diagrams 1000a depicting determination of a hygiene category related to an activity performed by a user, according to an embodiment of the disclosure. A system receives wearable sensor data, environmental conditions, user smart phone data as an input at the user data collection engine. The input in the form of raw data is analyzed and processed to generate a Categorized Repository of User Data (CRUD) also referred as hygiene data as an output used by other engines.

FIG. 10B illustrates a use case diagrams 1000b depicting determination of a type of the activity performed by a user, according to an embodiment of the disclosure. The system utilizes preprocessed data, the hygiene data, and the hygiene category to determine an identified hygiene category as an output.

FIG. 10C illustrates a use case diagrams 1000c depicting determination of an impact on personal hygiene of a user, according to an embodiment of the disclosure. The system utilizes the hygiene data, the identified hygiene category, identified type of activity and user personalized data as an input to calculate a change in a personal hygiene and an impact on the personal hygiene as an output.

FIG. 10D illustrates a use case diagrams 1000d depicting determination of one or more possible suggestions for maintaining the personal hygiene of the user, according to an embodiment of the disclosure. The system utilizes the hygiene data, the identified hygiene category, identified type of activity, and the impact on personal hygiene as an input to generate one or more possible suggestions as an output.

FIG. 10E illustrates a use case diagrams 1000e depicting determination of one or more suggestions from the one or more suggestions for maintaining the personal hygiene of the user, according to an embodiment of the disclosure. The system utilizes the hygiene data, the identified hygiene category, identified type of activity, and the impact, user personalized data, feedback, one or more possible suggestions to generate time-appropriate personalized PH suggestions also referred as one or more suggestions as an output.

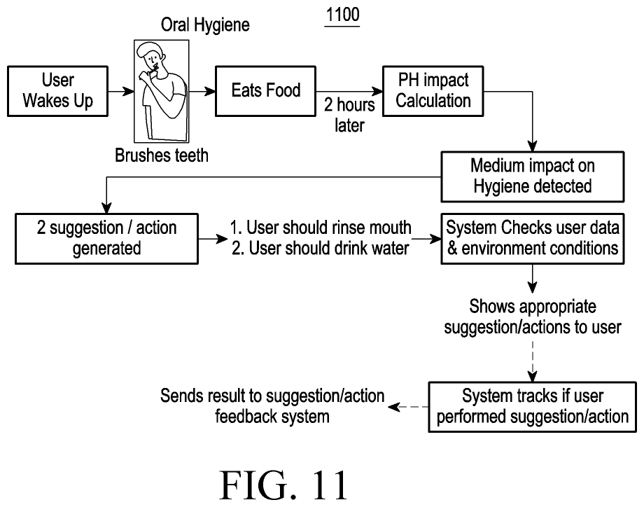

FIG. 11 illustrates a use case scenario 1100 for suggesting one or more suggestions to maintain oral hygiene to a user, according to an embodiment of the disclosure. The user performs a number of types of activities such as brushing teeth, eating food, or the like. Based on that, an impact level is calculated, and the one or more suggestions are provided to the user as depicted in Table 2.

TABLE 2

| | Sub-activity | | |
|---|---|---|---|
| User | Conditions | PH impact | Suggestions |
| User 1 | Brushing⇒Smoking⇒Eating | HIGH | 1. User should rinse mouth |
| | User 1 is in office | | 2. User should brush teeth |
| | | | 3. User should wash hands |
| | | | 4. User should use Listerine |
| User 2 | Brushing⇒Smoking⇒Eating | HIGH | 1. User should rinse mouth |
| | User 2 is in office | | 2. User should brush teeth |
| | | | 3. User should wash hands |
| | | | 4. User should use Listerine |
| User 3 | Didn't brush since morning | MEDIUM | 1. User should use mouth freshener |
| | User 3 is outside | | 2. User should drink water |
| User 4 | Didn't brush since morning | LOW | 1. User should drink water |
| | User 4 is outside | | |
| User 5 | Brushing⇒Eating | MEDIUM | 1. User should rinse mouth |
| | User 5 is at home | | 2. User should drink water |
| User 6 | Brushing⇒Eating | MEDIUM | 1. User should rinse mouth |
| | User 6 is at home | | 2. User should drink water |

Table 2 depicts one or more suggestions, condition associated with a type of activity, and an impact level related to oral hygiene

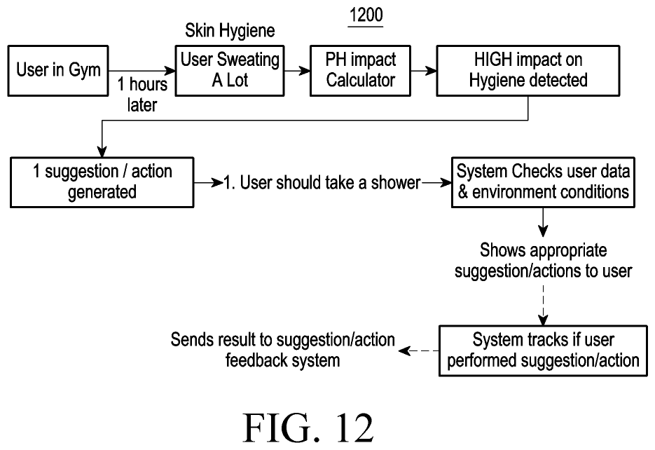

FIG. 12 illustrates a use case scenario 1200 for suggesting one or more suggestions to maintain skin hygiene to a user, according to an embodiment of the disclosure. The user performs a number of types of activities such as working out in a gym, sweating or the like. Based on that, an impact level is calculated, and the one or more suggestions are provided to the user as depicted in Table 3.

TABLE 3

| | | PH | |
|---|---|---|---|
| User | Sub-activity Conditions | impact | Suggestions |
| User 1 | Exercising⇒Sweating | HIGH | 1. User should take a proper bath |
| | User 1 is exercising for 1 | | 2. User should change clothes |
| | hour | | |
| User 2 | Exercising⇒Sweating | MEDIUM | 1. User should dry themselves |
| | User 2 is exercising for 1 | | 2. User should drink water |
| | hour | | |
| User 3 | Change in Skin | MEDIUM | 1. User should drink water |
| | Conductance ⇒ Dryness | | 2. User should apply moisturizer |
| | User 3 is at home (indoor) | | |
| User 4 | Change in Skin | LOW | 1. User should drink water |
| | Couductance ⇒ Dryness | | 2. User should apply moisturizer |
| | User 4 is at home (indoor) | | |
| User 5 | High Sun Exposure | HIGH | 1. User should apply 50 SPF sunscreen |
| | User 5 is roaming on a | | 2. User should use umbrella |
| | beach (Outdoor) | | |
| User 6 | High Sun Exposure | HIGH | 1. User should apply 50 SPF sunscreen |
| | User 6 is roaming on a | | 2. User should use umbrella |
| | beach (Outdoor) | | |

Table 3 depicts one or more suggestions, condition associated with a type of activity, and an impact level related to skin hygiene

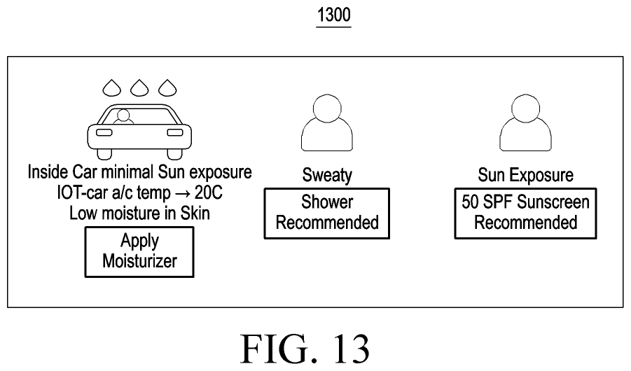

FIG. 13 illustrates a use case scenario 1300 for suggesting one or more suggestions to maintain skin hygiene, according to an embodiment of the disclosure. A user may go out and get exposed to sun or stay inside in an Air Conditioned (AC) environment, skin of the user may experience one or more changes. The skin may get sweaty or may get dry due to low moisture. As a skin hygiene level of the user is getting tracked, appropriate measures are suggested such as applying sunscreen of a specific SPF, applying a moisturizer, taking a shower or the like.

In an embodiment, the disclosure may be configured to manage a hygiene routine of a user suffering from depression. Users with depression may face challenges in maintaining daily hygiene routine. Skipping such activities over a long period may cause problems for the users hindering a treatment of existing issues with the users. The disclosure may be helpful for the user to maintain a healthy hygiene which in turn may better their depression at a faster rate.

In an embodiment, the disclosure may further be configured to educate/remind food handlers about personal hygiene. Bacteria that cause food poisoning may be everywhere and may be spread from one person to another and to the food if the person touches their nose, mouth, hair or your clothes, and then the food. The proposed subject matter may be useful to remind and suggest the person handling food to maintain personal hygiene levels.

The disclosure may further be configured to educate children about personal hygiene. For kids, good personal hygiene may ensure a good health. Well-kept hygiene prevents illnesses and assists in build a child's self-awareness. The disclosure solution may give reminders and suggestions to keep check on personal hygiene levels of children.

The disclosure may also be configured to keeping a check on sanitization levels of personal belongings. The disclosure includes a method to notify a user when personal belongings of the user are exposed to any dirt/dust to clean and sanitize them.

The disclosure also may be configured to assist elderly and medical patients to maintain their personal hygiene. The disclosure may can assist the elderly and medical patients in taking care of themselves by providing constant reminders and suggestions about their well-being like a personal support available to them.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A method for providing, by an electronic device, one or more suggestions for maintaining personal hygiene of a user, the method comprising:
receiving hygiene data associated with the user from at least one source in response to detecting an activity;
determining a hygiene category of the activity among a plurality of hygiene categories based on the hygiene data, and preprocessed hygiene data associated with the user;
determining a type of the activity among a plurality of types of activities based on the hygiene data and the hygiene category;
determining an impact level of the activity on the personal hygiene of the user based on the hygiene category, and the type of the activity; and
providing one or more suggestions for maintaining the personal hygiene to the user based on the impact level, the hygiene data, the hygiene category, and the activity.

2. The method of claim 1, further comprising:
determining whether the one or more suggestions is performed by the user; and
generating a feedback based on the determination.

3. The method of claim 1, wherein the at least one source comprises at least one of at least one sensor and memory of the electronic device.

4. The method of claim 1, wherein the hygiene data comprises at least one of:
sensor data received from at least one sensor;
user data associated with one or more of a reminder and an alarm associated with performing one or more of the plurality of types of activities set up by the user in the electronic device;
environmental data including at least of a temperature, a humidity, and precipitation, associated with a vicinity of the user;
pre-stored personal hygiene data associated with the user;
routine data generated by analyzing a daily routine of the user;
location associated with the user at a time of occurrence of the activity; and
contextual data generated from a combination of at least two of the sensor data, the user data, the environmental data, the pre-stored personal hygiene data, and the routine data.

5. The method of claim 1, wherein the plurality of hygiene categories comprises at least one of an oral hygiene category, a skin hygiene category, a hair hygiene category, a sanitization hygiene category, and a combination of at least two of the oral hygiene category, the skin hygiene category, the hair hygiene category, and the sanitization hygiene category.

6. The method of claim 1, wherein the plurality of types of activities comprises at least one of brushing teeth, smoking, flossing, sanitizing, washing hands, drinking, eating, rinsing mouth, bathing, receiving sun exposure, sweating, going out in a plurality of weather conditions, wearing a headgear, removing a headgear, sneezing, coughing, performing a plurality of hand activities, and washing hair.

7. The method of claim 1, wherein determining the hygiene category comprises:
determining a similarity score between the hygiene data and the preprocessed hygiene data;
generating a plurality of weight-hygiene pairs by pairing the similarity score with a weight associated with the respective hygiene category amongst the plurality of hygiene categories;
calculating a confidence score associated with a corresponding hygiene category based on the plurality of weight-hygiene pairs; and
selecting the hygiene category from the plurality of hygiene categories with a highest confidence score.

8. The method of claim 1, wherein determining the type of the activity comprises:
correlating the hygiene data, the hygiene category, the preprocessed hygiene data with one another by applying a Machine Learning (ML) technique.

9. The method of claim 1, wherein determining the impact level comprises:
assigning an activity weight to the activity and a duration weight to a time duration of the activity;
calculating an activity score based on the activity weight, the duration weight, a previous activity score associated with a previous activity, and a weight associated with the previous activity score, wherein the weight associated with the previous activity score is based on a time duration between occurrence of the previous activity and the activity; and
determining the impact level by comparing the activity score with the previous activity score, wherein the impact level is one of a low impact level, a medium impact level, and a high impact level.

10. The method of claim 1, wherein providing the one or more suggestion comprises:
identifying one or more possible suggestions among a plurality of pre-stored possible suggestions in a database based on the hygiene data, the hygiene category, the type of activity, the impact level, wherein the plurality of pre-stored possible suggestions is mapped with the plurality of hygiene categories and the plurality of types of activities; and
identifying the one or more suggestions from the one or more possible suggestions based on the hygiene data, the impact level, one or more previous suggestions suggested to the user, and feedback associated with usage of one or more previous suggestions suggested to the user.

11. The method of claim 1, wherein the one or more suggestions comprises at least one of brushing teeth, using a mouthwash, using a mouth freshener, rinsing mouth with water, drinking water, washing hands, taking a bath, washing hair, applying hair products to hair, combing hair, changing clothes, using a towel to dry one or more body parts, applying skincare products, using an umbrella, sanitizing hands, wearing a face mask, and wearing gloves.

12. An electronic device for providing one or more suggestions for maintaining personal hygiene of a user, the electronic device comprising:
memory storing one or more computer programs; and
one or more processors communicatively coupled to the memory,
wherein the one or more computer programs include computer-executable instructions that, when executed by the one or more processors individually or collectively, cause the electronic device to:
receive hygiene data associated with the user from at least one source in response to detecting an activity,
determine a hygiene category of the activity among a plurality of hygiene categories based on the hygiene data, and preprocessed hygiene data associated with the user,
determine a type of the activity among a plurality of types of activities based on the hygiene data and the hygiene category,
determine an impact level of the activity on the personal hygiene of the user based on the hygiene category, and the type of the activity, and
provide one or more suggestions for maintaining the personal hygiene to the user based on the impact level, the hygiene data, the hygiene category, and the activity.

13. The electronic device of claim 12, wherein the one or more computer programs further include computer-executable instructions that, when executed by the one or more processors individually or collectively, cause the electronic device to:
determine whether the one or more suggestions is performed by the user, and
generate a feedback based on the determination.

14. The electronic device of claim 12, wherein the at least one source comprises at least one of at least one sensor and the memory of the electronic device.

15. The electronic device of claim 12, wherein the hygiene data comprises at least one of:
sensor data received from at least one sensor;
user data associated with one or more of a reminder and an alarm associated with performing one or more of the plurality of types of activities set up by the user in the electronic device;
environmental data including at least of a temperature, a humidity, and precipitation, associated with a vicinity of the user;
pre-stored personal hygiene data associated with the user;
routine data generated by analyzing a daily routine of the user;
location associated with the user at a time of occurrence of the activity; and
contextual data generated from a combination of at least two of the sensor data, the user data, the environmental data, the pre-stored personal hygiene data, and the routine data.

16. The electronic device of claim 12, wherein the plurality of hygiene categories comprises at least one of an oral hygiene category, a skin hygiene category, a hair hygiene category, a sanitization hygiene category, and a combination of at least two of the oral hygiene category, the skin hygiene category, the hair hygiene category, and the sanitization hygiene category.

17. The electronic device of claim 12, wherein the plurality of types of activities comprises at least one of brushing teeth, smoking, flossing, sanitizing, washing hands, drinking, eating, rinsing mouth, bathing, receiving sun exposure, sweating, going out in a plurality of weather conditions, wearing a headgear, removing a headgear, sneezing, coughing, performing a plurality of hand activities, and washing hair.

18. The electronic device of claim 12, wherein the one or more suggestions comprises at least one of brushing teeth, using a mouthwash, using a mouth freshener, rinsing mouth with water, drinking water, washing hands, taking a bath, washing hair, applying hair products to hair, combing hair, changing clothes, using a towel to dry one or more body parts, applying skincare products, using an umbrella, sanitizing hands, wearing a face mask, and wearing gloves.

19. One or more non-transitory computer-readable storage media storing one or more computer programs including computer-executable instructions that, when executed by one or more processors of an electronic device individually or collectively, cause the electronic device to perform operations, the operations comprising:
receiving hygiene data associated with a user of the electronic device from at least one source in response to detecting an activity;
determining a hygiene category of the activity among a plurality of hygiene categories based on the hygiene data, and preprocessed hygiene data associated with the user;
determining a type of the activity among a plurality of types of activities based on the hygiene data and the hygiene category;
determining an impact level of the activity on personal hygiene of the user based on the hygiene category, and the type of the activity; and
providing one or more suggestions for maintaining the personal hygiene to the user based on the impact level, the hygiene data, the hygiene category, and the activity.

20. The one or more non-transitory computer-readable storage media of claim 19, the operations further comprising:
determining whether the one or more suggestions is performed by the user; and
generating a feedback based on the determination.
